(19) United States
(12) Patent Application Publication
(10) Pub. No.: US 2015/0276703 A1
(43) Pub. Date: Oct. 1, 2015

(54) INFORMATION PROCESSING APPARATUS, INFORMATION PROCESSING METHOD, AND STORAGE MEDIUM

(71) Applicant: FUJI XEROX CO., LTD., Tokyo (JP)

(72) Inventors: Toshihiro NUMAUCHI, Kanagawa (JP); Masanori YOSHIZUKA, Kanagawa (JP)

(73) Assignee: FUJI XEROX CO., LTD., Tokyo (JP)

(21) Appl. No.: 14/665,269

(22) Filed: Mar. 23, 2015

(30) Foreign Application Priority Data
Mar. 27, 2014 (JP) 2014-066289

(51) Int. Cl. G01N 33/44; G01B 21/08; G01B 21/20; G06T 19/20

(52) U.S. Cl. CPC G01N 33/442; G06T 19/20; G01B 21/08; G01B 21/20; G06T 2219/004; G06T 2219/012

(57) ABSTRACT

An information processing apparatus includes an inspecting unit that performs a mold requirement inspection on three-dimensional information for manufacturing a product expressed by the three-dimensional information; and an outputting unit that outputs information for plotting a line indicative of an inexpedient part on a surface of the product expressed by the three-dimensional information, as information indicative of an inspection result by the inspecting unit.

### CROSS-REFERENCE TO RELATED APPLICATIONS

This application is based on and claims priority under 35 USC 119 from Japanese Patent Application No. 2014-066289 filed Mar. 27, 2014.

### BACKGROUND

The present invention relates to an information processing apparatus, an information processing method, and a storage medium.

### SUMMARY

According to an aspect of the invention, there is provided an information processing apparatus including an inspecting unit that performs a mold requirement inspection on three-dimensional information for manufacturing a product expressed by the three-dimensional information; and an outputting unit that outputs information for plotting a line indicative of an inexpedient part on a surface of the product expressed by the three-dimensional information, as information indicative of an inspection result by the inspecting unit.

### BRIEF DESCRIPTION OF THE DRAWINGS

An exemplary embodiment of the present invention will be described in detail based on the following figures, wherein:

FIG. 1 is a conceptual module configuration diagram for a configuration example of an exemplary embodiment;

FIG. 2 is an explanatory illustration showing a system configuration example to which this exemplary embodiment is applied;

FIG. 3 is a flowchart showing a processing example according to this exemplary embodiment;

FIG. 4 is an explanatory illustration showing an example of a screen that is used for an inspection request;

FIG. 5 is an explanatory illustration showing an example of a screen that displays an inspection result;

FIG. 6 is an explanatory illustration showing an example of a screen for three-dimensionally displaying an inspection result;

FIG. 7 is an explanatory illustration showing an example of combinations of display forms for inspection items;

FIG. 8 is an explanatory view showing a display example of an inspection result;

FIG. 9 is an explanatory view showing a display example of an inspection result;

FIGS. 10A and 10B are explanatory views showing a display example of an inspection result;

FIG. 11 is an explanatory view showing a display example of an inspection result;

FIG. 12 is an explanatory view showing a display example of an inspection result;

FIG. 13 is an explanatory view showing a display example of an inspection result;

FIG. 14 provides explanatory views showing a display example of an inspection result; and

FIG. 15 is a block diagram showing a hardware configuration example of a computer that implements the exemplary embodiment.

### DETAILED DESCRIPTION

An exemplary embodiment desirable for implementing the invention is described below with reference to the drawings.

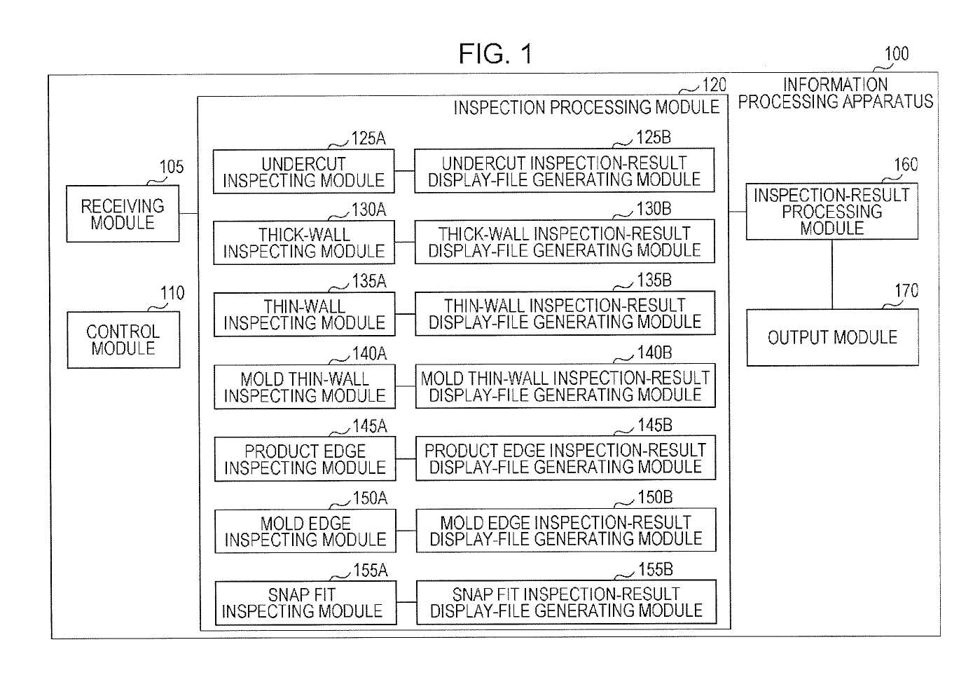

FIG. 1 is a conceptual module configuration diagram showing a configuration example according to this exemplary embodiment.

A module represents a component, such as software (a computer program) or hardware, which may be generally logically separated. Hence, a module in this exemplary embodiment represents not only a module being a computer program, but also a module being a hardware configuration. Therefore, description in this exemplary embodiment also involves a computer program that causes a computer to function as such a module (a program that causes a computer to execute respective steps, a program that causes a computer to function as respective units, and a program that causes a computer to provide respective functions), a system, and a method. For convenience of description, wordings “store,” “cause . . . to store,” and other wordings equivalent thereto are used. These wordings represent causing a memory to store . . . or controlling a memory to store . . . in the case in which the exemplary embodiment is a computer program. Also, modules may correspond to functions one by one. However, when being mounted or installed, a single module may be formed of a single program, plural modules may be formed of a single program, or a single module may be formed of plural programs. Also, plural modules may be executed by a single computer, or a single module may be executed by plural computers in a distributed or parallel environment. A single module may include other module. Also, “connection” is used for physical connection, and logical connection (for example, transmission and reception of data, an instruction, or reference relationship among data). An expression “predetermined” represents being determined before target processing. The situation includes a situation before processing according to this exemplary embodiment is started, and a situation even after processing according to this exemplary embodiment is started as long as the situation is before target processing. In other words, the expression “predetermined” is used as being determined in accordance with the condition and state of the current situation, or the condition and state of the previous situation. If there are plural “predetermined values,” the values may be different, or two or more values (of course, all the values) may be the same. Also, an expression “if A, do B” is used as “determining whether A or not, and doing B if it is determined as A,” unless otherwise the determination whether A or not is not required.

Also, a system or an apparatus includes a case in which a system or an apparatus is formed of plural computers, hardware, plural devices, etc., connected by a communication measure such as a network (including communication connection in a one-to-one correspondence), and a case in which a system or an apparatus is provided by a single computer, hardware, a single device, etc. “An apparatus” and “a system” are used as mutually equivalent words. Of course, “a system” does not include a social “scheme” (social system) that is merely an artificial agreement.

Also, target information is read from a memory every processing of each module or every processing if plural steps of processing are executed in a module, and after the processing, the processing result is written out to the memory. Hence, the description of reading from the memory before the processing and writing out to the memory after the processing may be occasionally omitted. In this case, a memory may include a hard disk, a random access memory (RAM), an external storage medium, a memory arranged via a communication line, and a register in a central processing unit (CPU).

An information processing apparatus 100 according to this exemplary embodiment outputs the result of an inspection including an inspection for an undercut on three-dimensional information (mold requirement inspection, hereinafter, also merely referred to as “inspection”). As shown in an example in FIG. 1, the information processing apparatus 100 includes a receiving module 105, a control module 110, an inspection processing module 120, an inspection-result processing module 160, and an output module 170.

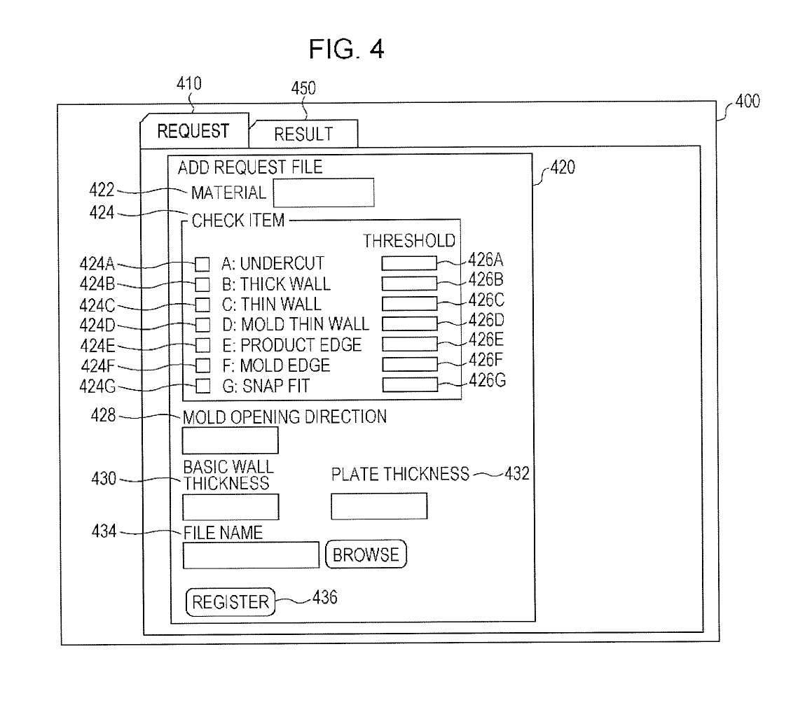

The receiving module 105 is connected to the inspection processing module 120. The receiving module 105 receives at least three-dimensional information being an inspection target. The three-dimensional information is data generated by three-dimensional computer aided design (CAD) (including intermediate data, and compatible data for transfer to other CAD), and is, for example, a file in a format of Parasolid or the like. The three-dimensional information is for creating a mold (for example, metal mold) that generates a product expressed by the three-dimensional information. Hence, an object expressed by three-dimensional information may be “a product that is generated by a mold” or “a mold.” If the target object is a product that is generated by a mold, the object is called “a product expressed by three-dimensional information,” and if the target object is a mold, the object is called “a mold expressed by three-dimensional information.” When three-dimensional information is received, the three-dimensional information itself may be received, or a file name of a file that is stored in a memory (file server or the like) accessible to the information processing apparatus 100 may be designated (designation by an operation of a user like an example in FIG. 4, described later). Information that is received by the receiving module 105 (that is, information input by the user) requires only three-dimensional information, and other information may be optional. Such optional information may be automatically detected from the three-dimensional information or a predetermined value may be used if such optional information is not input. For example, automatic detection of a mold opening direction may use a technology disclosed in related art.

Also, the three-dimensional information that is received by the receiving module 105 may not include history information indicative of the history of operations for creating the three-dimensional information. For example, only information for forming a three-dimensional shape may be included. That is, the inspection processing module 120 performs an inspection only on the basis of the three-dimensional information. Of course, the three-dimensional information that is received by the receiving module 105 may include history information indicative of the history of operations for creating the three-dimensional information.

Further, the receiving module 105 may receive the three-dimensional information, and type information indicative of the type of a mold expressed by three-dimensional information. The type information includes material information indicative of the material of a product to be manufactured with the mold. The type may include, for example, plastic and pressing. In addition, types for forging, casting, die-casting, glass, rubber, etc. may be provided. Such a type may be previously determined, or the user may select a type like the example shown in FIG. 4 (described later). In this case, each inspecting module in the inspection processing module 120 performs an inspection based on the material information received by the receiving module 105.

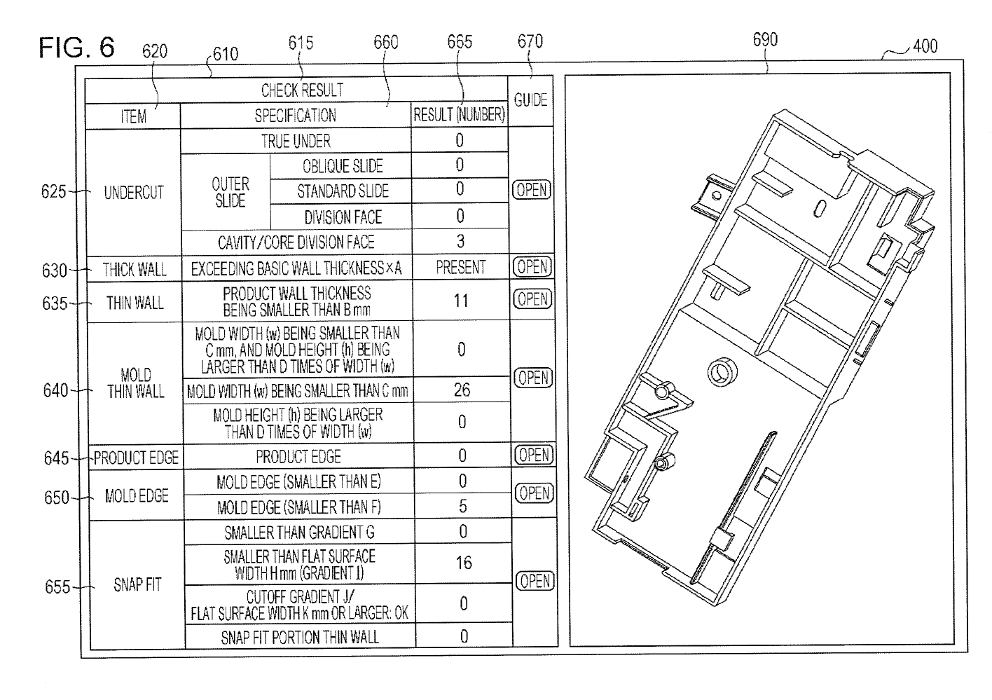

Further, the receiving module 105 may receive an inspection item to be performed by the inspection processing module 120. The inspection item may be, for example, an undercut, a thick wall, a thin wall, a mold thin wall, a product edge, a mold edge, and a snap fit. Also, specification items may be provided for these items (specification column 660 shown in an example in FIG. 6, described later). Such items may be previously determined, or the user may select any of the items like the example shown in FIG. 4 (described later). In this case, each inspecting module in the inspection processing module 120 performs an inspection corresponding to the inspection item received by the receiving module 105.

Further, the receiving module 105 may receive a value (including a threshold etc.) corresponding to the inspection item to be performed by the inspection processing module 120. The value to be used for an inspection of each inspection item may be, for example, a value relating to a basic wall thickness when the material is plastic (for example, a value by which the basic wall thickness is multiplied (to be specific, in case of a thick wall, when it is defined that “a portion with a value that is X times larger than the basic wall thickness (for example, X=1.2) or larger is an inexpedient part,” X serves as a threshold, of course, the threshold X being able to be changed)), and a value of plate thickness if the material is for pressing. The threshold corresponding to an inspection item may be one or plural. The threshold may be previously determined, or the user may select a threshold like the example shown in FIG. 4 (described later). In this case, each inspecting module in the inspection processing module 120 performs an inspection for the inspection item received by the receiving module 105 in accordance with the threshold. The basic wall thickness may be automatically detected from the three-dimensional information. The method of automatic detection may include, for example, calculating the wall thickness of each surface of the received three-dimensional information and using a statistical value. The statistical value may use, for example, the most frequent vale of the wall thickness (the value of wall thickness that is the most frequently set), or a mean value of the thickest portion and the thinnest portion.

Further, the receiving module 105 may receive a mold opening direction expressed by three-dimensional information (a removal direction when a product is removed from a mold, an open/close direction of the mold). The mold opening direction may be the up/down direction, the left/right direction, the front/rear direction, an oblique direction, etc., with respect to a predetermined surface of a product expressed by three-dimensional information. The mold opening direction may be previously determined, or the user may select the mold opening direction like the example shown in FIG. 4 (described later). In this case, each inspecting module in the inspection processing module 120 performs an inspection in accordance with the mold opening direction received by the receiving module 105. As described above, the mold opening direction may be also automatically detected from the three-dimensional information. In this case, if the receiving module 105 does not receive information of the mold opening direction (if the user does not input), the mold opening direction may be automatically detected. That is, the inspection processing module 120 specifies the mold opening direction from the three-dimensional information received by the receiving module 105, and each inspecting module (such as an undercut inspecting module 125A) may perform an inspection based on the specified mold opening direction. To be specific, the received three-dimensional information is analyzed, information of the opening direction and the basic wall thickness is calculated, and the inspection is performed on the basis of the calculation result. In this case, the three-dimensional information may be only three-dimensional shape data indicative of a form. If the three-dimensional shape data is analyzed, the operation history of CAD etc. is no longer required, and compatibility is enhanced. Also, the above-described information may be estimated from information that is input and received, in addition to the shape data.

Also, even when the receiving module 105 receives the information of the mold opening direction, the mold opening direction may be automatically detected from the three-dimensional information. If both pieces of information (the received information of the mold opening direction and the automatically detected information of the mold opening direction) are different, an alarm for the difference may be displayed to allow the user to select the information. Information other than the mold opening direction may be treated similarly.

The control module 110 controls the entire information processing apparatus 100. For example, the control module 110 causes the inspection processing module 120 to perform processing in accordance with information received by the receiving module 105. To be specific, when an inspection item is received by the receiving module 105, the control module 110 selects a module in the inspection processing module 120, the module which corresponds to the inspection item, and causes the module to perform inspection processing. When a value is received by the receiving module 105, the control module 110 causes each module in the inspection processing module 120, the module which makes response in accordance with the value, to perform inspection processing. Also, when the mold opening direction expressed by the three-dimensional information is received by the receiving module 105, the control module 110 causes each module in the inspection processing module 120, the module which makes response in accordance with the received mold opening direction, to perform inspection processing.

Also, the control module 110 may store an inspection item and an inspection parameter (values including a threshold etc.) in association with user information (including login information etc.), and when the receiving module 105 receives three-dimensional information, the inspection item and the inspection parameter associated with the user information may be applied to cause each inspecting module to perform an inspection.

The inspection processing module 120 is connected to the receiving module 105 and the inspection-result processing module 160, and performs an inspection for manufacturing a product expressed by three-dimensional information received by the receiving module 105. The inspection processing module 120 includes an undercut inspecting module 125A, an undercut inspection-result display-file generating module 125B, a thick-wall inspecting module 130A, a thick-wall inspection-result display-file generating module 130B, a thin-wall inspecting module 135A, a thin-wall inspection-result display-file generating module 135B, a mold thin-wall inspecting module 140A, a mold thin-wall inspection-result display-file generating module 140B, a product edge inspecting module 145A, a product edge inspection-result display-file generating module 145B, a mold edge inspecting module 150A, a mold edge inspection-result display-file generating module 150B, a snap fit inspecting module 155A, and a snap fit inspection-result display-file generating module 155B. It is to be noted that the inspection processing module 120 may at least include the undercut inspecting module 125A and the undercut inspection-result display-file generating module 125B. Also, as an inspection performed by the inspection processing module 120, an inspection that is performed by at least one pair of the pair of the thick-wall inspecting module 130A and the thick-wall inspection-result display-file generating module 130B, the pair of the thin-wall inspecting module 135A and the thin-wall inspection-result display-file generating module 135B, the pair of the mold thin-wall inspecting module 140A and the mold thin-wall inspection-result display-file generating module 140B, the pair of the product edge inspecting module 145A and the product edge inspection-result display-file generating module 145B, the pair of the mold edge inspecting module 150A and the mold edge inspection-result display-file generating module 150B, and the pair of the snap fit inspecting module 155A and the snap fit inspection-result display-file generating module 155B may be additionally performed.

The undercut inspecting module 125A is connected to the undercut inspection-result display-file generating module 125B. The thick-wall inspecting module 130A is connected to the thick-wall inspection-result display-file generating module 130B. The thin-wall inspecting module 135A is connected to the thin-wall inspection-result display-file generating module 135B. The mold thin-wall inspecting module 140A is connected to the mold thin-wall inspection-result display-file generating module 140B. The product edge inspecting module 145A is connected to the product edge inspection-result display-file generating module 145B. The mold edge inspecting module 150A is connected to the mold edge inspection-result display-file generating module 150B. The snap fit inspecting module 155A is connected to the snap fit inspection-result display-file generating module 155B.

The undercut inspecting module 125A performs inspection processing for an undercut on three-dimensional information. An undercut represents a recessed or protruding shape that is not removed from a mold merely by pushing the shape in the open/close direction of the mold when a molded product is removed from a mold. Undercut processing requires an undercut processing mechanism, such as an outer slide or an inclined pin. If a product has an undercut portion, the portion is formed by another portion (slide core) and moved every time when the mold is opened and closed, so as not to interrupt removal of the product. However, such a mold may typically have a complicated structure, may be expensive, and may result in a failure during molding. Therefore, it is desirable to design a product without an undercut portion. Hence, the undercut inspecting module 125A performs inspection processing whether the three-dimensional information received by the receiving module 105 has an undercut or not. The inspection processing for the presence of an undercut may use a technology disclosed in related art. For example, as a true under and an outer slide, (a) oblique slide, (b) standard slide, (c) division face, and the number and positions of Cavity/Core division faces are calculated. To be specific, inspection items for an undercut include (1) under portion, and (2) division face. (1) Under portion is for inspecting whether an undercut portion is present or not, to avoid that undercut processing is required and a mold structure becomes complicated. (2) Division face is for inspecting whether a portion that avoids an undercut by dividing a face by Cavity/Core/Standard slide is present or not, to avoid discrimination between a true undercut and an oblique slide/inner slide portion.

If an inexpedient part is detected by the undercut inspecting module 125A, the undercut inspection-result display-file generating module 125B generates inexpedient information for three-dimensionally expressing the inexpedient part. For example, a display file that may be three-dimensionally displayed and that is configured to provide displaying for instructing an undercut portion is generated.

The thick-wall inspecting module 130A performs inspection processing for a thick wall on three-dimensional information.

A thick wall and a thin wall are described. Resin has a volume that is changed between a molten state and a solid state. In general, resin is contracted when solidified as compared with the molten state. The ratio of the change is called contraction ratio. Also, immediately after a molded product is removed from a mold, the temperature of the product is higher than the normal temperature, and reaches the normal temperature for the first time after several hours or several tens of hours elapse. At this time, the molded product is contracted as being cooled. Such a difference in dimension of the molded product is called molding contraction. The amount of molding contraction varies basically depending on the type of the plastic material, and also depending on the shape of the molded product and the molding condition. The mold is required to be made with a large size with regard to the amount of molding contraction. In case of a thermoplastic plastic, a crystalline plastic has a markedly larger value than that of a non-crystalline plastic. Also, the value of a material filled with glass fiber is typically decreased although the value is changed depending on the type of filling material or reinforcing material, or the contained amount.

There may be findings for the relationship between the shape of a product and the gate design of a mold as follows. When a molding material flows into a cavity through a gate of a mold, an orientation of a resin or a filling material that forms the molding material appears. The orientation may be changed depending on the shape of a product or how the gate is arranged. A directivity also appears in the value of molding contraction ratio. Also, the directivity may cause deformation, such as warping or twisting, to occur in the product. To prevent this, the gate shape of the mold has to be improved. In the gate design, if the size (cross-sectional area) of the gate is increased, the molding contraction ratio typically tends to be decreased.

Next, the relationship with the wall thickness of the product is described. If the wall thickness of the molded product is increased, even though the same type of molding material is used, a sink mark likely appears on the surface. If the wall thickness of a molded product is increased, the molding contraction ratio typically tends to be increased. If the product is thin-walled, the flow distance of the product is decreased (propagation of a pressure is decreased), possibly resulting in short molding. Also, the number of gates may be increased for filling. If a thick-wall portion is partially present, cooling is not uniformly performed among the inside of the thick-wall portion, the surface-layer surface, and the portion with the basic wall thickness, possibly resulting in deformation, such as a sink mark or warping. Hence, the thick-wall inspecting module 130A performs inspection processing whether three-dimensional information received by the receiving module 105 has a thick wall or not. The inspection processing for the presence of a thick wall may use a technology disclosed in related art. For example, the number and positions of portions each having a wall thickness exceeding a basic wall thickness×A (A being a predetermined value, for example, 1.2) are calculated. To be specific, an inspection item for the thick wall may be (1) thick wall. (1) Thick wall is for inspecting whether a thick wall is present or not in the entire product as compared with the basic wall thickness to avoid a molding defect (such as sink mark or warping).

If an inexpedient part is detected by the thick-wall inspecting module 130A, the thick-wall inspection-result display-file generating module 130B generates inexpedient information for three-dimensionally expressing the inexpedient part. For example, a display file that may be three-dimensionally displayed and that is configured to provide displaying for instructing a thick-wall portion is generated.

The thin-wall inspecting module 135A performs inspection processing for a thin wall on three-dimensional information. As described above, a filling failure (short molding) likely occurs in a thin-wall portion. Hence, the thin-wall inspecting module 135A performs inspection processing whether the three-dimensional information received by the receiving module 105 has a thin wall or not. The inspection processing for the presence of a thin wall may use a technology disclosed in related art. For example, the number and positions of portions each having a product wall thickness smaller than B (B being a predetermined value, for example, 1 mm) are calculated. To be specific, an inspection item for the thin wall may be (1) smaller-than-1-mm thin wall. (1) Smaller-than-1-mm thin wall is for inspecting whether a thin-wall portion with 1 mm or smaller is present or not, to avoid a molding failure (short shot or the like).

If an inexpedient part is detected by the thin-wall inspecting module 135A, the thin-wall inspection-result display-file generating module 135B generates inexpedient information for three-dimensionally expressing the inexpedient part. For example, a display file that may be three-dimensionally displayed and that is configured to provide displaying for instructing a thin-wall portion is generated.

The mold thin-wall inspecting module 140A performs inspection processing for a mold thin wall on three-dimensional information.

A mold thin wall is described.

When a mold structure portion is narrow and long, the mold structure portion is likely broken by a pressure during molding. Hence, product design is required to be made so that the mold does not have a shape, such as a circular (or ellipsoidal) pin shape portion with h≧α×φd (h being a length of a narrow part of the pin, and φd being a diameter of the narrow part of the pin), or a plate pin shape portion with h≧β×a (h being a length of a narrow part of the pin, and a being a length of a short side of an upper surface of the pin). It is to be noted that α and β are predetermined values. For example, α is 5, and β is 4. It is assumed that the minimum values of d and a are predetermined values (for example, 1 mm). Hence, the mold thin-wall inspecting module 140A performs inspection processing whether the three-dimensional information received by the receiving module 105 has a mold thin wall or not. The inspection processing for the presence of a mold thin wall may use a technology disclosed in related art. For example, the number and positions of (1) a portion with a mold width being smaller than C (C being a predetermined value, for example, 1 mm) and a mold height being larger than D (D being a predetermined value, for example, 4) times of a mold width, (2) a portion with a mold width being smaller than C, and (3) a portion with a mold height being larger than D times of a mold width. To be specific, an inspection item for the mold thin wall may be (1) mold thin wall. (1) Mold thin wall is for inspecting whether a portion with a thin wall (smaller than 1 mm) is present or not as the mold shape and whether a portion with a height (h) being larger than four times of a width (W) is present or not (h/w>4) as the mold shape to avoid insufficiency in mold intensity.

If an inexpedient part is detected by the mold thin-wall inspecting module 140A, the mold thin-wall inspection-result display-file generating module 140B generates inexpedient information for three-dimensionally expressing the inexpedient part. For example, a display file that may be three-dimensionally displayed and that is configured to provide displaying for instructing a mold thin-wall portion is generated.

The product edge inspecting module 145A performs inspection processing for a product edge on three-dimensional information.

A product edge is an item to avoid a filling failure (short molding) of the above-described thin-wall portion. Hence, the product edge inspecting module 145A performs inspection processing whether the three-dimensional information received by the receiving module 105 has a product edge or not. The inspection processing for the presence of a product edge may use a technology disclosed in related art. For example, the number and positions of product edge portions are calculated. To be specific, an inspection item for the product edge may be (1) product edge. (1) Product edge is for inspecting whether an edge portion is present or not in the product shape to avoid that direct engraving is unable to be performed during mold machining (nesting division (telescopic division), electric discharge machining).

If an inexpedient part is detected by the product edge inspecting module 145A, the product edge inspection-result display-file generating module 145B generates inexpedient information for three-dimensionally expressing the inexpedient part. For example, a display file that may be three-dimensionally displayed and that is configured to provide displaying for instructing a product edge portion is generated.

The mold edge inspecting module 150A performs inspection processing for a mold edge on three-dimensional information.

A mold edge is a check item for ensuring the mold intensity similarly to the above-described mold thin wall. Hence, the mold edge inspecting module 150A performs inspection processing whether the three-dimensional information received by the receiving module 105 has a mold edge or not. The inspection processing for the presence of a mold edge may use a technology disclosed in related art. For example, the number and positions of (1) a portion of a mold edge (with an angle smaller than E (E being a predetermined value, for example, 60) degrees), and (2) a portion of a mold edge (with an angle smaller than F (F being a predetermined value, for example, 89) degrees). To be specific, an inspection item for the mold edge may be (1) mold edge. (1) Mold edge is for inspecting whether an edge portion is present or not in the mold shape to avoid insufficiency in mold intensity.

If an inexpedient part is detected by the mold edge inspecting module 150A, the mold edge inspection-result display-file generating module 150B generates inexpedient information for three-dimensionally expressing the inexpedient part. For example, a display file that may be three-dimensionally displayed and that is configured to provide displaying for instructing a mold edge portion is generated.

The snap fit inspecting module 155A performs inspection processing for a snap fit on three-dimensional information.

A snap fit is described.

A cutoff is a portion at which nests (telescopic parts) are directly mated with each other in a moving direction of a mold. The nests are mated while sliding on each other every time when the mold is opened and closed. If the mating state is loose, blur is generated. If the mating state is too tight, a fatigue failure occurs in the mold. Hence, a cutoff mating adjustment process is required. If there are many cutoff faces, the mold manufacturing period tends to be long. Owing to this, the design has to be made to decrease the number of cutoff faces. To avoid interference of the mold and to decrease wear of the mold, a cutoff face has to have an angle of a predetermined value (for example, 3 degrees) or larger. Hence, the snap fit inspecting module 155A performs inspection processing whether the three-dimensional information received by the receiving module 105 has a snap fit (cutoff shape) or not. The inspection processing for the presence of a snap fit may use a technology disclosed in related art. For example, the number and positions of snap fit thin-wall portions each having a gradient smaller than a gradient G (G being a predetermined value, for example, 5) degrees, a flat surface width smaller than a flat surface width H (H being a predetermined value, for example, 1 mm) (gradient I (I being a predetermined value, for example, 5) degrees), and a cutoff gradient J (J being a predetermined value, for example, 5) degrees/a flat surface width K (K being a predetermined value, for example, 1 mm) or larger are calculated. To be specific, inspection items for the snap fit may be (1) snap fit inspection and (2) snap-fit-shaped thin wall inspection. (1) snap fit inspection is for inspecting whether cutoff gradient/flat surface width of a snap fit portion is ensured or not to avoid insufficiency in mold intensity. (2) Snap-fit-shaped thin wall inspection is for inspecting the presence of a portion with a height (h) being larger than four times of a width (w) (h/w>4) as the mold shape of the snap fit portion to avoid insufficiency in mold intensity.

If an inexpedient part is detected by the snap fit inspecting module 155A, the snap fit inspection-result display-file generating module 155B generates inexpedient information for three-dimensionally expressing the inexpedient part. For example, a display file that may be three-dimensionally displayed and that is configured to provide displaying for instructing a snap fit portion is generated.

The inspection-result processing module 160 is connected to the inspection processing module 120 and the output module 170. The inspection-result processing module 160 associates an inspection result by the inspection processing module 120 with each inspection item and information for three-dimensionally expressing an inexpedient part in the inspection item.

Also, the inspection-result processing module 160 may output information for plotting a line indicative of an inexpedient part (for example, an undercut portion) on a surface of a product expressed by three-dimensional information, as information indicative of an inspection result by the inspection processing module 120. The “line” is, of course, distinguishable from a line that plots a product expressed by the three-dimensional information. For example, the line may be in an arrow form, a line with a different thickness, a line with a different color, or a dotted line, as compared with the line that plots the product expressed by the three-dimensional information.

Further, the inspection-result processing module 160 may output information for plotting a surface being an inexpedient part of a product expressed by three-dimensional information in a manner distinguishable from a surface not being an inexpedient part.

Further, the inspection-result processing module 160 may output information for plotting a line indicative of an inexpedient part on a surface of a product expressed by three-dimensional information if the inspection processing module 120 performs an inspection for any of a thick wall, a thin wall, a product edge, and a mold edge.

Further, the inspection-result processing module 160 may output information for plotting a surface being an inexpedient part in a manner distinguishable from a surface not being an inexpedient part, and information for plotting a line indicative of an inexpedient part on a surface of the product expressed by the three-dimensional information.

Also, the inspection-result processing module 160 may output information for plotting a line indicative of an inexpedient part in a space configured of a surface of a product expressed by three-dimensional information, as information indicative of an inspection result by the inspection processing module 120.

“A space configured of a surface” may be a space configured of plural surfaces (for example, a space in which two surfaces face each other, or a space surrounded by three surfaces), and a space configured of a single surface (for example, a line with a start point and an end point on a single surface).

Further, the inspection-result processing module 160 may output information for plotting a surface being an inexpedient part of a product expressed by three-dimensional information in a manner distinguishable from a surface not being an inexpedient part.

Further, the inspection-result processing module 160 may output information for plotting a line indicative of an inexpedient part in a space configured of a surface of a product expressed by three-dimensional information if the inspection processing module 120 performs an inspection for any of an undercut, a mold thin wall, and a snap fit.

Further, the inspection-result processing module 160 may output information for plotting a surface being an inexpedient part of a product expressed by three-dimensional information in a manner distinguishable from a surface not being an inexpedient part, and information for plotting a line indicative of an inexpedient part in a space configured of a surface of the product expressed by the three-dimensional information, if the inspection processing module 120 performs an inspection for any of an undercut and a mold thin wall.

The output module 170 is connected to the inspection-result processing module 160. The output module 170 outputs inexpedient information and an inspection item in an associated manner. Also, the output module 170 may output three-dimensional information for displaying including an inexpedient part by using three-dimensional shape information received by the receiving module 105 and inexpedient information. Outputting information includes, for example, displaying information on a display device such as a display, transmitting information to other information processing apparatus through a communication line, writing information in a memory, and storing information in a storage medium such as a memory card.

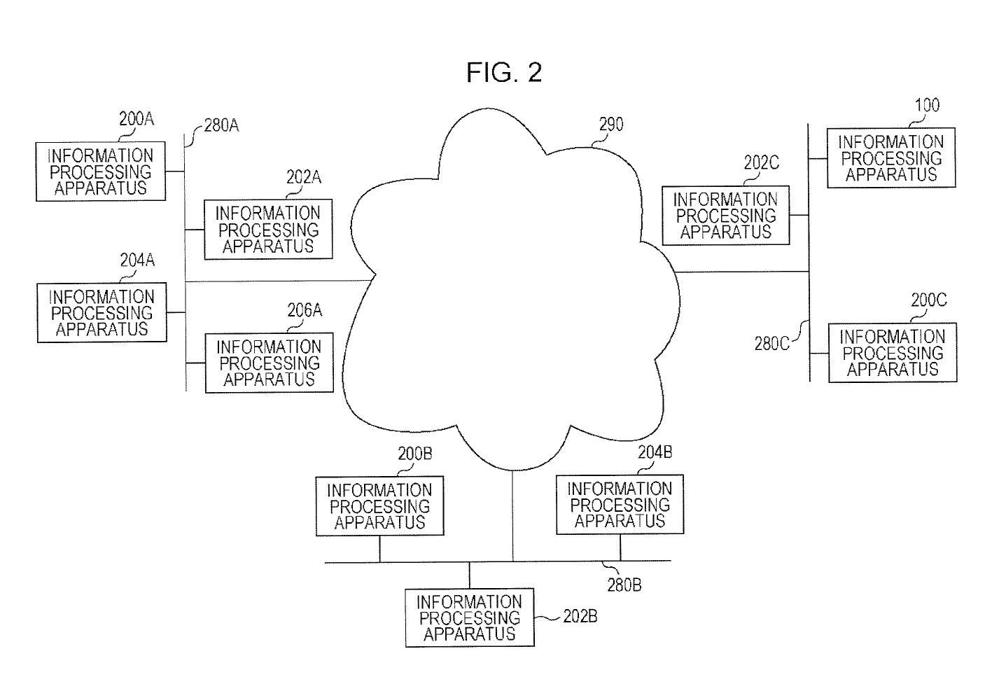

FIG. 2 is an explanatory illustration showing a system configuration example to which this exemplary embodiment is applied.

A communication line 290 connects a communication line 280A, a communication line 280B, and a communication line 280C, and is, for example, the Internet serving as a communication infrastructure. The communication line 280A, the communication line 280B, and the communication line 280C each connect an information processing apparatus 200 in each group, and are each, for example, an intranet serving as a communication infrastructure constructed in a company.

An information processing apparatus 200A, an information processing apparatus 202A, an information processing apparatus 204A, an information processing apparatus 206A, an information processing apparatus 200B, an information processing apparatus 202B, an information processing apparatus 204B, an information processing apparatus 200C, and an information processing apparatus 202C are connected to the information processing apparatus 100 through the communication line 280A, the communication line 280B, the communication line 280C, or the communication line 290. The information processing apparatus 100 provides inspection processing for three-dimensional information as so-called cloud service (a design support system including a non-moldable portion detecting function). The information processing apparatus 200 has installed therein a Web browser. The information processing apparatus 200 gives three-dimensional information to the information processing apparatus 100, gives an instruction for an inspection, receives the inspection result from the information processing apparatus 100, and displays the inspection result by three-dimensional displaying.

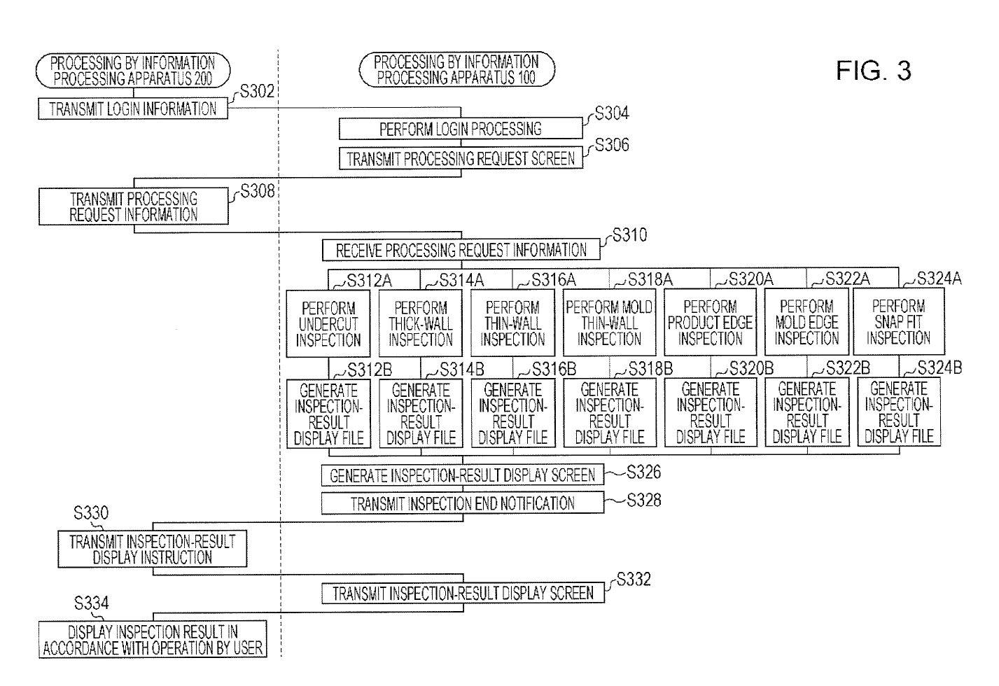

FIG. 3 is a flowchart showing a processing example according to this exemplary embodiment. FIG. 3 shows a processing example executed between the information processing apparatus 100 and the information processing apparatus 200.

In step S302, the information processing apparatus 200 transmits login information. For example, a combination of a user ID and a password, information within an IC card owned by a user, biometric information such as fingerprint information are transmitted as the login information to the information processing apparatus 100. This login is for using a service of inspection processing according to this exemplary embodiment.

In step S304, the control module 110 performs login processing. The login processing is performed on the basis of the login information transmitted in step S302. For example, it may be determined whether or not the combination of the user ID and the password matches information stored in the information processing apparatus 100. If login is failed, a message indicative of the login failure is transmitted to the information processing apparatus 200.

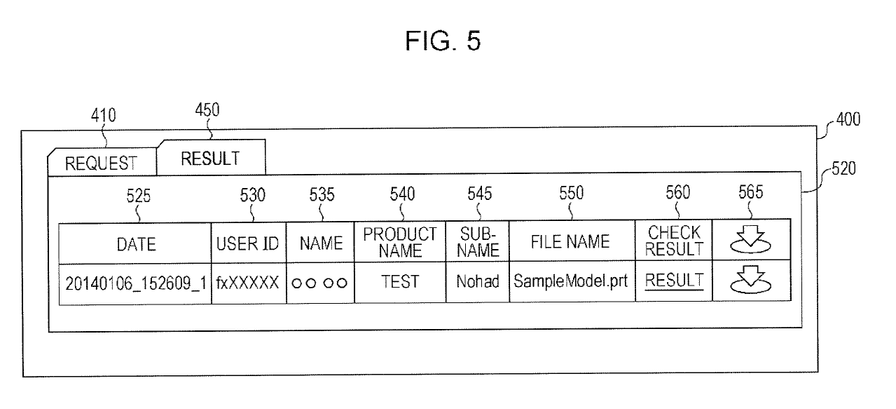

In step S306, the control module 110 transmits a processing request screen. For example, a file (HTML document etc.) for displaying the content of a screen 400 exemplarily shown in FIG. 4 is transmitted. FIG. 4 is an explanatory illustration showing an example of the screen 400 that is used for an inspection request. A request tab 410 is an example of the processing request screen. A result tab 450 is a screen that displays the inspection result corresponding to the processing request as described later by using an example in FIG. 5.

The request tab 410 includes a request reception region 420. Displayed in the request reception region 420 are a material column 422, a check item column 424, a mold opening direction column 428, a basic wall thickness column 430, a plate thickness column 432, a file name column 434, and a register button 436.

The material column 422 is a pull-down menu for designating whether three-dimensional information being an inspection target is for plastic or pressing. In addition, menus for forging, casting, die-casting, glass, rubber, and other menu may be provided.

Displayed in the check item column 424 for setting an item desired to be inspected by the user and for setting a threshold are an undercut check column 424A, a thick-wall check column 424B, a thin-wall check column 424C, a mold thin-wall check column 424D, a product edge check column 424E, a mold edge check column 424F, a snap fit check column 424G, an undercut threshold column 426A, a thick-wall threshold column 426B, a thin-wall threshold column 426C, a mold thin-wall threshold column 426D, a product edge threshold column 426E, a mold edge threshold column 426F, and a snap fit threshold column 426G. The undercut threshold column 426A corresponds to the undercut check column 424A, the thick-wall threshold column 426B corresponds to the thick-wall check column 424B, the thin-wall threshold column 426C corresponds to the thin-wall check column 424C, the mold thin-wall threshold column 426D corresponds to the mold thin-wall check column 424D, the product edge threshold column 426E corresponds to the product edge check column 424E, the mold edge threshold column 426F corresponds to the mold edge check column 424F, and the snap fit threshold column 426G corresponds to the snap fit check column 424G. Each threshold column 426 may be omitted, or plural threshold columns may be provided. As a default setting, all items may be checked or a predetermined item (for example, the undercut check column 424A) may be checked. Also, a default value may be input in each threshold column 426, or a range allowable as a threshold may be set. If a value outside the range is input, an error message may be displayed.

The mold opening direction column 428 is for designation of a mold opening direction expressed by three-dimensional information. This column is a pull-down menu for designating one of, for example, the up/down direction, the left/right direction, the front/rear direction, and an oblique direction, with respect to a predetermined surface in a product expressed by the three-dimensional direction as described above. Alternatively, a vector serving as the opening direction may be designated by the user. The vector designation may use, for example, extraction of a normal vector of a selected designation surface, and extraction of a vector component connecting two selected and designated points.

The basic wall thickness column 430 is displayed when plastic is selected in the material column 422, and is a type of the value received by the receiving module 105. The plate thickness column 432 is displayed when pressing is selected in the material column 422, and is a type of the above-described threshold. A default value may be input in the basic wall thickness column 430 or the plate thickness column 432, or a range allowable as a threshold may be set. If a value outside the range is input, an error message may be displayed.

The file name column 434 is for uploading three-dimensional information, and is for designating a file name of a file having the three-dimensional information as the content. Also, the three-dimensional information may be directly uploaded from CAD. As described above, the columns other than the file name column 434 indicative of the three-dimensional information do not have to be provided. Hence, no column may be provided except the file name column 434, or some columns of these may remain. Also, the user does not have to input a value in a column other than the file name column 434.

In step S308, the information processing apparatus 200 transmits processing request information. Describing with reference to FIG. 4, when the register button 436 is selected by the user, at this time, the content of the column such as the material column 422 is transmitted to the information processing apparatus 100.

In step S310, the receiving module 105 receives the processing request information.

In step S312A, the undercut inspecting module 125A performs undercut inspection processing on the three-dimensional information received by the receiving module 105.

In step S312B, the undercut inspection-result display-file generating module 125B generates an inspection-result display file.

In step S314A, the thick-wall inspecting module 130A performs thick-wall inspection processing on the three-dimensional information received by the receiving module 105.

In step S314B, the thick-wall inspection-result display-file generating module 130B generates an inspection-result display file.

In step S316A, the thin-wall inspecting module 135A performs thin-wall inspection processing on the three-dimensional information received by the receiving module 105.

In step S316B, the thin-wall inspection-result display-file generating module 135B generates an inspection-result display file.

In step S318A, the mold thin-wall inspecting module 140A performs mold thin-wall inspection processing on the three-dimensional information received by the receiving module 105.

In step S318B, the mold thin-wall inspection-result display-file generating module 140B generates an inspection-result display file.

In step S320A, the product edge inspecting module 145A performs product edge inspection processing on the three-dimensional information received by the receiving module 105.

In step S320B, the product edge inspection-result display-file generating module 145B generates an inspection-result display file.

In step S322A, the mold edge inspecting module 150A performs mold edge inspection processing on the three-dimensional information received by the receiving module 105.

In step S322B, the mold edge inspection-result display-file generating module 150B generates an inspection-result display file.

In step S324A, the snap fit inspecting module 155A performs snap fit inspection processing on the three-dimensional information received by the receiving module 105.

In step S324B, the snap fit inspection-result display-file generating module 155B generates an inspection-result display file.

The processing of steps S312A, S314A, . . . , and S324A may be performed in parallel or successively. Also, processing may be performed by using the result of other processing (including intermediate result).

In step S326, the inspection-result processing module 160 generates an inspection-result display screen. For example, a file for three-dimensionally displaying the inspection result in step S312A or other step is generated.

In step S328, the output module 170 transmits an inspection end notification. For example, a notification may be made by using an e-mail or other method to the user who made the inspection processing request. At this time, a download target (URL or the like) of the file generated in step S326 may be included in the content of the e-mail or other method

In step S330, the information processing apparatus 200 transmits an inspection-result display instruction.

In step S332, the output module 170 transmits an inspection-result display screen. For example, a file (HTML document etc.) for displaying the content of an inspection-processing notification region 520 exemplarily shown in FIG. 5 is transmitted. FIG. 5 is an explanatory illustration showing an example of the screen 400 for displaying the inspection result. The inspection-processing notification region 520 is a screen example when the result tab 450 is selected.

Displayed in the inspection-processing notification region 520 are a date column 525, a user ID column 530, a name column 535, a product name column 540, a sub-name column 545, a file name column 550, a check result column 560, and a download instruction column 565. In the date column 525, a date of inspection processing (year, month, day, hour, minute, second, and unit shorter than second, or a combination of these) is displayed. In the name column 535, the user ID of a user who made an inspection request is displayed. In the name column 535, the name of the user is displayed. In the product name column 540 and the sub-name column 545, a product name and a sub-name of three-dimensional information being an inspection target are displayed. In the file name column 550, a file name of the three-dimensional information being the inspection target is displayed. In the check result column 560, URL for displaying the inspection result with the Web browser is displayed. When this column is selected by the user, a screen exemplarily shown in FIG. 6 (described later) is displayed. In the download instruction column 565, an icon for download of data of the inspection result is displayed. For example, a file for displaying a screen exemplarily shown in FIG. 6 (viewer data) is allowed to be downloaded. Alternatively, a file of the three-dimensional information (the file indicated in the file name column 550, CAD data) being the inspection target may be allowed to be downloaded.

The product name column 540 and the sub-name column 545 may be omitted. If the product name column 540 and the sub-name column 545 are added, a product name column and a sub-name column may be added to the screen 400 exemplarily shown in FIG. 4 to perform processing by using the product name and the sub-name. For example, a request item using the screen 400 and an inspection result etc. for the request may be stored as a log, and the log may be transmitted to a person relating to the product or sub in addition to the user who made the request (to be specific, an address (an e-mail address, etc.) of a person who performed extraction by using a table that stores an administrator or the like associated with the product or sub.

In step S334, the information processing apparatus 200 displays the inspection result in accordance with an operation by the user. The display form is described with reference to examples in FIGS. 6 to 14.

FIG. 6 is an explanatory illustration showing an example of the screen 400 for three-dimensionally displaying an inspection result. In the screen 400, a check result table 610 and a 3D display region 690 are displayed.

The check result table 610 displays an inspection result in a table form, and includes a check result column 615 and a guide column 670. The check result column 615 includes an item column 620, a specification column 660, and a result (number) column 665.

In the item column 620, inspection items are displayed. These inspection items correspond to the inspection results by the inspecting modules in the inspection processing module 120, and correspond to the items checked in the check item column 424 shown in the example in FIG. 4. As the item column 620, at least one of an undercut column 625, a thick wall column 630, a thin wall column 635, a mold thin wall column 640, a product edge column 645, a mold edge column 650, and a snap fit column 655 is displayed. At least the undercut column 625 may be displayed.

In the specification column 660, specification items for the respective inspection items are displayed. In the result (number) column 665, the inspection results (presence, the number of inexpedient parts, etc.) for the respective items in the specification column 660 are displayed. As the undercut column 625, (1) true under; (2a) oblique slide, (2b) standard slide, and (2c) division face for (2) outer slide; and (3) Cavity/Core division face are displayed according to the inspection result by the undercut inspecting module 125A. As the thick wall column 630, a portion (1) exceeding basic wall thickness×A (for example, 1.1, 1.2, 1.3, or 1.4) according to the inspection result by the thick-wall inspecting module 130A is displayed. As the thin wall column 635, a portion with (1) product wall thickness being smaller than B (for example, 1, 2, or 3 mm) according to the inspection result by the thin-wall inspecting module 135A is displayed. As the mold thin wall column 640, a portion with (1) mold width being smaller than C mm (for example, 1, 2, or 3 mm) and mold height being larger than D (for example, 3, 4, 5, or 6) times of width, a portion with (2) mold width being smaller than C mm, and a portion with (3) mold height being larger than D times of width according to the inspection result of the mold thin-wall inspecting module 140A are displayed. As the product edge column 645, a portion with (1) a product edge according to the inspection result by the product edge inspecting module 145A is displayed. As the mold edge column 650, (1) mold edge (smaller than E (for example, 55, 60, 65, or 70) degrees), and (2) mold edge (smaller than F (for example, 86, 87, 88, 89, 90, 91, or 92) degrees according to the inspection result by the mold edge inspecting module 150A are displayed. As the snap fit column 655, a portion with a gradient (1) smaller than gradient G (for example, 3, 4, 5, 6, or 7) degrees, a flat surface width (2) smaller than flat surface width H (for example, 1, 2, or 3 mm) (gradient I (for example, 3, 4, 5, 6, or 7) degrees), (3) cutoff gradient J (for example, 3, 4, 5, 6, or 7) degrees/flat surface width K (for example, 1, 2, or 3 mm) or larger, and a portion of (4) snap fit portion thin wall, according to the inspection result by the snap fit inspecting module 155A are displayed.

In the guide column 670, an “open” button is displayed for displaying explanation of the corresponding inspection item. If the “open” button in each inspection item is selected, the inspection item, and explanation of a method for addressing the inexpedient part etc. are displayed by using a pop-up screen.

In the 3D display region 690, the product expressed by the three-dimensional information being the inspection target is three-dimensionally displayed. Three-dimensional rotation, enlargement, reduction, two-dimensional cross-sectional display, etc., are performed in accordance with an operation by the user. Then, an inexpedient part is displayed in a different manner from the other portion (a portion not being an inexpedient part) as the inspection result.

If an inexpedient part is present as the inspection result, at least one item of the item column 620, the specification column 660, and the result (number) column 665 or a combination of these may be displayed in a different form to be distinguished from an item without an inexpedient part. For example, the background of the item column 620 for an inspection item without an inexpedient part may be light green, and the background of the item column 620 for an inspection item with an inexpedient part may be red. Instead of the color or in addition to the color, the thickness of a line, the shape of a line (dotted line, solid line, etc.), the pattern, or animation display may be changed in a different manner. For example, an inspection item with an inexpedient part may be displayed by flashing.

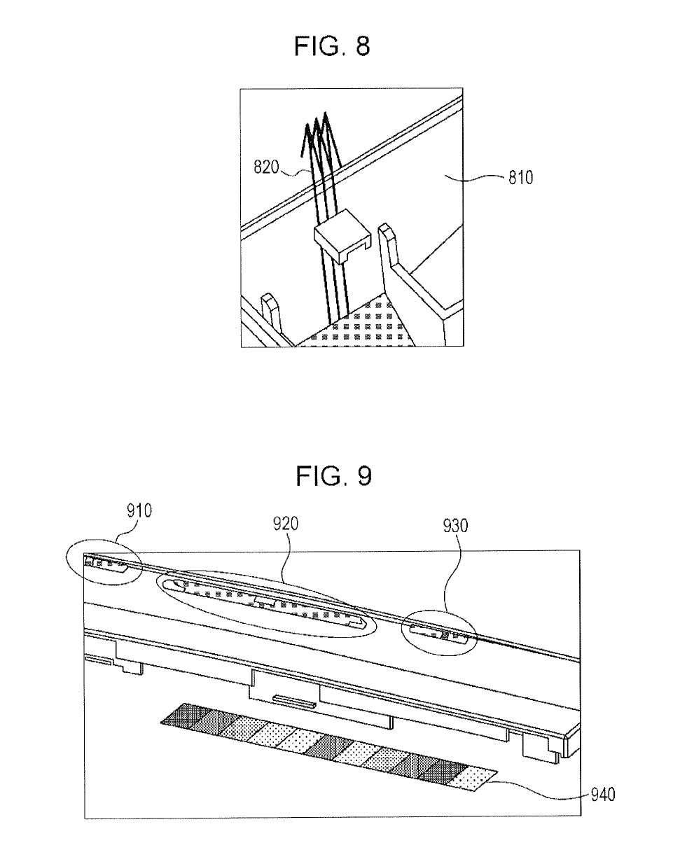

If an item (any one of respective items of the item column 620, the specification column 660, and the result (number) column 665) with an inexpedient part is selected by an operation of the user, the corresponding inexpedient part may be three-dimensionally displayed in the 3D display region 690. That is, since each inspection item in the check result table 610 is associated with the corresponding inexpedient part in the 3D display region 690, if each inspection item in the check result table 610 is selected, the corresponding inexpedient part of the inspection item is displayed in the 3D display region 690. For example, when a link in the undercut column 625 of the item column 620 is clicked, the corresponding inexpedient part colored in a three-dimensionally displayed model in the 3D display region 690 is able to be recognized. The display method of each inspection item is described later with reference to examples in FIGS. 8 to 14.

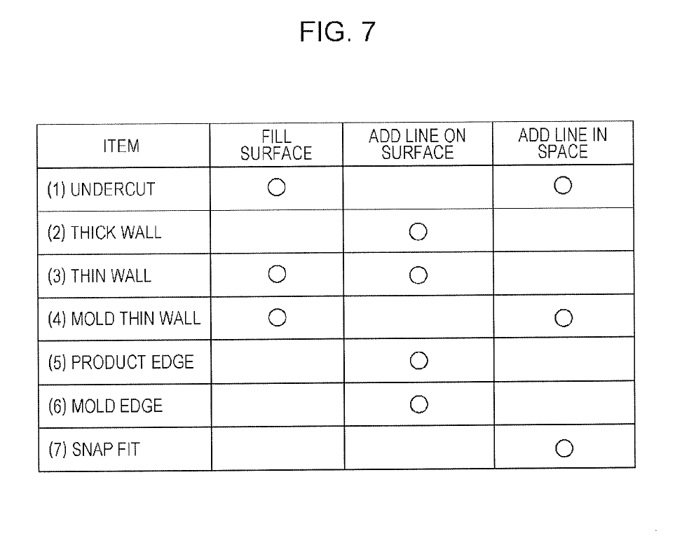

FIG. 7 is an explanatory illustration showing an example of combinations of display forms for inspection items.

A table shown in the example in FIG. 7 shows a display form of an inexpedient part when each inspection item is three-dimensionally displayed. That is, in (1) undercut, “fill surface” and “add line in space” are performed, in (2) thick wall, “add line on surface” is performed, in (3) thin wall, “fill surface” and “add line on surface” are performed, in (4) mold thin wall, “fill surface” and “add line in space” are performed, in (5) product edge, “add line on surface” is performed, in (6) mold edge, “add line on surface” is performed, and in (7) snap fit, “add line in space” is performed. “Fill surface” represents, if a surface of a product (or a mold) expressed by three-dimensional information itself is an inexpedient part, filling the surface with a color different from the color of the other surfaces. “Add line on surface” represents plotting a line at a portion on a surface where a defect occurs. The line is not limited to a linear shape, such as a straight line, a curved line, or an arrow, and may be a shape, such as a character or a symbol. Also, the line may be colored. “Add line in space” represents plotting a line in “a space configured of a surface” as described above.

The expression of the inspection result may be selectively used as follows:

(1) surface filling: when only an entire surface is required to be instructed in three-dimensional information for displaying;
(2) line on surface: when a portion on a surface is required to be instructed in three-dimensional information for displaying; and
(3) line in space: when a portion without an element of three-dimensional information for displaying is required to be instructed.

For example, in case of a thin wall, the portion of the thin wall is required to be instructed. Hence, expression is provided by a combination of surface filling and line on surface.

Expression for each of the other items is as follows.

### Undercut

To instruct a surface corresponding to an inexpedient part by the undercut inspection, surface filling is used. To instruct the position and direction of the under, line in space (arrow display) is used.

### Thick Wall

To instruct a portion corresponding to an inexpedient part by the thick wall inspection, line on surface is used. Surface filling may be also used. However, if the color types of lines to be displayed become larger than a predetermined number, surface filling may not be performed. If surface filling is additionally used, it may be difficult to view the display.

### Mold Thin Wall

To instruct a surface corresponding to an inexpedient part by the mold thin-wall inspection, surface filling is used. Also, line in space is used for instructing the portion. The corresponding portion is a portion that becomes a mold, and a portion where a molded product is not present.

### Product Edge/Mold Edge

To instruct an edge corresponding to an inexpedient part by the product edge/mold edge inspection, the edge is handled as line on surface. Since an end portion of a surface is an edge, line on surface is used.

### Snap Fit

To simply plot and instruct a mold shape of a portion corresponding to an inexpedient part by the snap fit inspection, line in space is used.

FIG. 8 is an explanatory view showing a display example of an inspection result. FIG. 8 shows a display example in the 3D display region 690 of an inspection result by the undercut inspecting module 125A. In this display example, black concern-portion display arrows (lines) 820 are displayed at an under portion of a real under surface 810. Arrows in plural directions may extend from the same portion.

FIG. 9 is an explanatory view showing a display example of an inspection result. FIG. 9 shows a display example in the 3D display region 690 of an inspection result by the thick-wall inspecting module 130A. In this display example, three inexpedient parts are present as the inspection result for the thick wall, and colored symbols “⊚” (double circles) (inexpedient-part display lines 910, 920, and 930) are plotted at thick-wall portions being inexpedient parts. For example, a color bar is displayed for reference. For example, the inspection is performed in a range from basic wall thickness×A mm to (basic wall thickness×A mm)+3 mm, and the result is displayed with a gradation color like an example 940 added thereto.

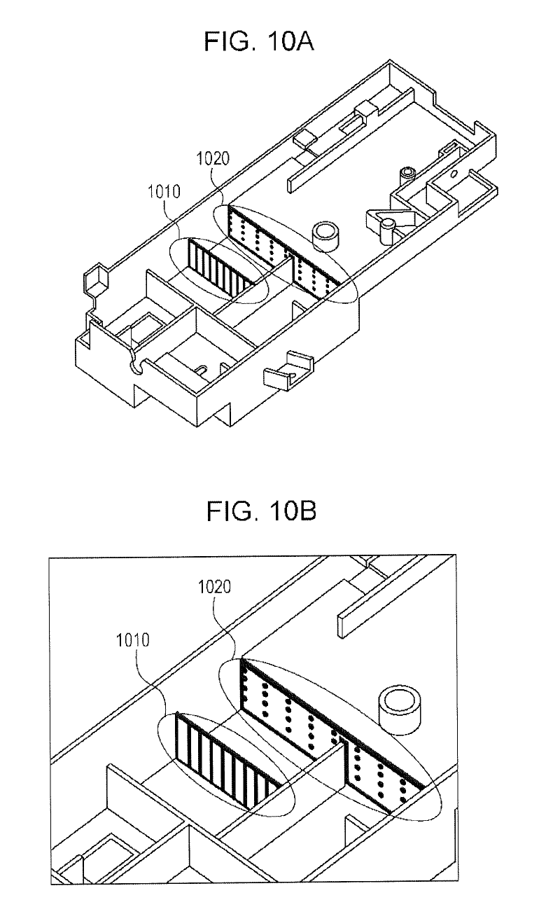

FIGS. 10A and 10B are explanatory views showing a display example of an inspection result. FIG. 10A shows a display example in the 3D display region 690 of the inspection result by the thin-wall inspecting module 135A. FIG. 10B is an enlarged view of FIG. 10A. In this display example, a red solid line is plotted at a portion 1010 smaller than B mm, and the surface is filled with pink color. Then, a blue dotted line is plotted at a portion 1020 smaller than B mm and equal to or larger than B1 (for example, 0.4, 0.5, 0.6, 0.7, or 0.8) mm, and the surface is filled with pink color.

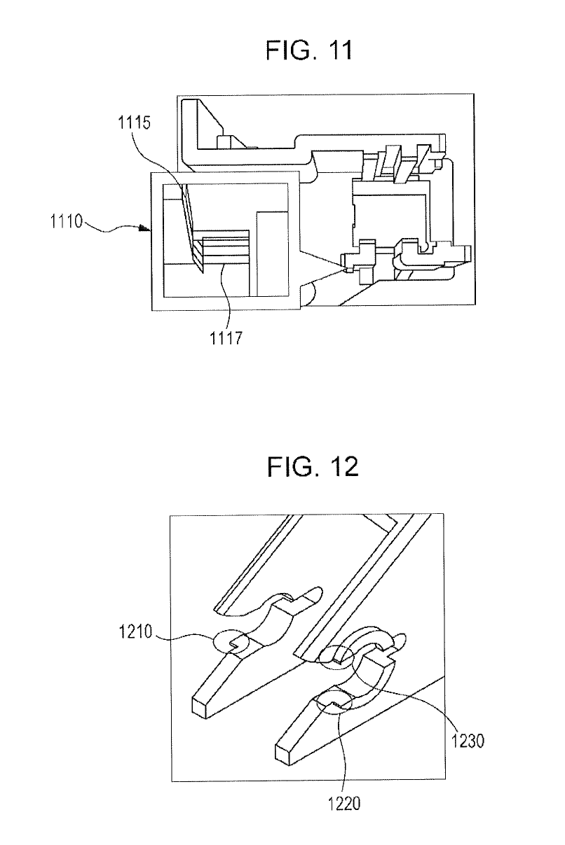

FIG. 11 is an explanatory view showing a display example of an inspection result. FIG. 11 shows a display example in the 3D display region 690 of an inspection result by the mold thin-wall inspecting module 140A. In this display example, a line is plotted at a portion with a width (w) of a mold being smaller than C mm, and a inexpedient-part display arrow (line) 1117 having arrows at both ends is plotted at a concern surface 1115 in an enlarged display region 1110.

FIG. 12 is an explanatory view showing a display example of an inspection result. FIG. 12 shows a display example in the 3D display region 690 of an inspection result by the product edge inspecting module 145A. In this display example, a line of magenta color is plotted at edges of inexpedient parts, like inexpedient-part display lines 1210, 1220, and 1230.

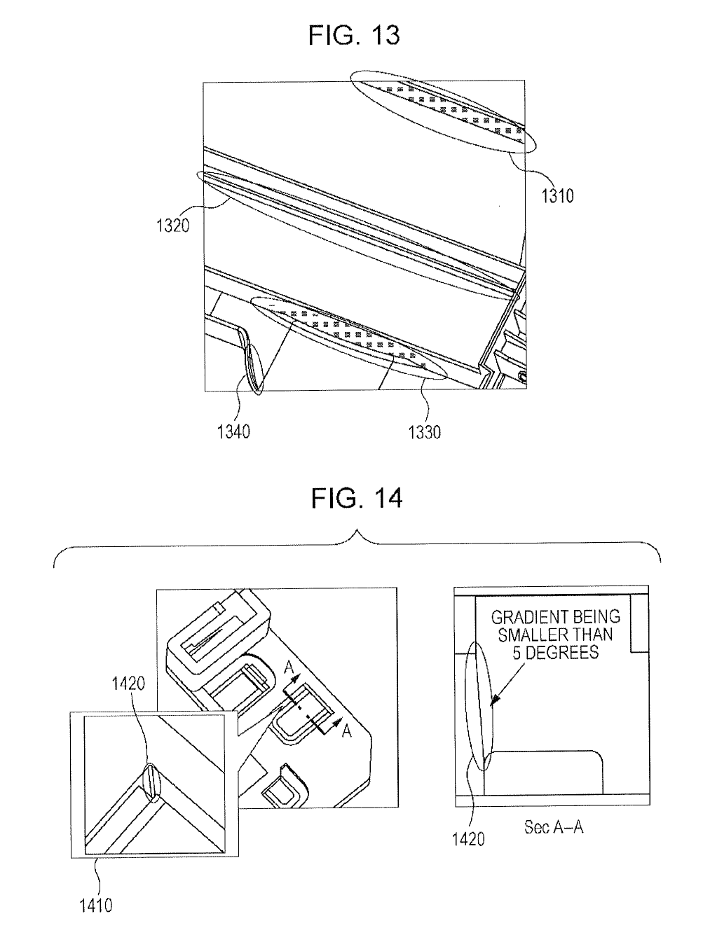

FIG. 13 is an explanatory view showing a display example of an inspection result. FIG. 13 shows a display example in the 3D display region 690 of an inspection result by the mold edge inspecting module 150A. In this display example, a line of magenta color is plotted when a mold edge of an inexpedient part is smaller than the mold edge E, and a line of green color is plotted when a mold edge of an inexpedient part is smaller than the mold edge F, like inexpedient-part display lines 1310, 1320, 1330, and 1340.

FIG. 14 provides explanatory views showing a display example of an inspection result. FIG. 14 shows a display example in the 3D display region 690 of an inspection result by the snap fit inspecting module 155A. In this display example, a snap fit portion in which a cutoff gradient is smaller than G degrees is recognized as an inexpedient part, and a line is plotted like an inexpedient-part display line 1420 in an enlarged display region 1410 as shown in the left figure in FIG. 14. Also, the right figure in FIG. 14 shows a cross-sectional surface taken along line A-A in the left figure in FIG. 14. In the right figure in FIG. 14, the line is plotted like the inexpedient-part display line 1420.

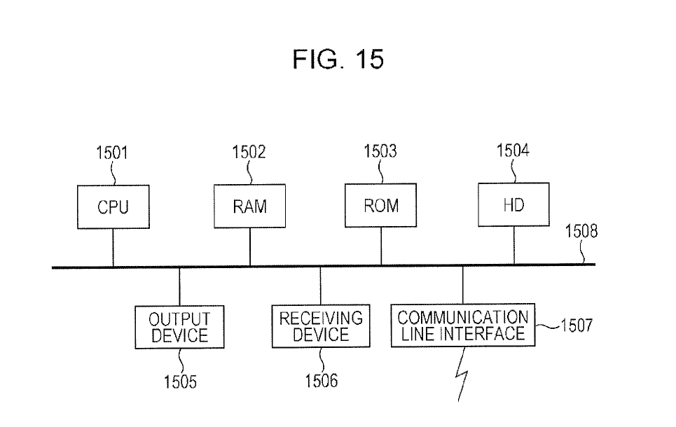

A hardware configuration of a computer that executes a program according to this exemplary embodiment is a typical computer as exemplarily shown in FIG. 15, and specifically a computer or the like that may serve as a personal computer or a server. That is, for specific example, a computer uses a CPU 1501 as a processing unit (an arithmetic operation unit), and a RAM 1502, a ROM 1503, and an HD 1504 as memories. For example, a hard disk may be used as the HD 1504. The computer includes the CPU 1501 that executes programs of, for example, the receiving module 105, the control module 110, the undercut inspecting module 125A, the undercut inspection-result display-file generating module 125B, the thick-wall inspecting module 130A, the thick-wall inspection-result display-file generating module 130B, the thin-wall inspecting module 135A, the thin-wall inspection-result display-file generating module 135B, the mold thin-wall inspecting module 140A, the mold thin-wall inspection-result display-file generating module 140B, the product edge inspecting module 145A, the product edge inspection-result display-file generating module 145B, the mold edge inspecting module 150A, the mold edge inspection-result display-file generating module 150B, the snap fit inspecting module 155A, the snap fit inspection-result display-file generating module 155B, the inspection-result processing module 160, and the output module 170; the RAM 1502 that stores the programs and data; the ROM 1503 that stores, for example, a program for booting this computer; the HD 1504 serving as an auxiliary memory (for example, flash memory); a receiving device 1506 that receives data in accordance with an operation by a user on a keyboard, a mouse, a touch panel, etc.; an output device 1505, such as a CRT or a liquid crystal display; a communication line interface 1507, such as a network interface card, for connection to a communication network; and a bus 1508 that connects the above-described units for transmission and reception of data. Multiple such computers may be connected through a network.

In the above-described exemplary embodiment, configurations provided by computer programs are implemented as the exemplary embodiment by causing a system with the hardware configuration to read the computer programs being software and by causing the software and hardware resources to cooperate to each other.

The hardware configuration shown in FIG. 15 is a mere configuration example. The configuration of this exemplary embodiment is not limited to the configuration shown in FIG. 15, and may be any configuration as long as the modules described in this exemplary embodiment may be executed. For example, a part of the modules may be formed of dedicated hardware (for example, application specific integrated circuit, ASIC, or the like), a part of the modules may be arranged in an external system and may be connected through a communication line, and further the system shown in FIG. 15 may be multiple systems mutually connected through a communication line and the multiple systems may operate in an associated manner. Alternatively, in particular, a part of the modules may be arranged in any of a copier, a facsimile, a scanner, a printer, and a multiple-function device (an image processing apparatus having at least two functions of a scanner, a printer, a copier, and a facsimile), instead of a personal computer.

Also, the above-described exemplary embodiment may be recognized as each of exemplary embodiments according to the invention as follows, or may be a combination of these exemplary embodiments. The exemplary embodiments are:

(A1) An information processing apparatus including:

a receiving unit that receives three-dimensional information including at least a three-dimensional shape;

an inspecting unit that performs a mold requirement inspection including at least an undercut on the three-dimensional information, as a mold requirement inspection for manufacturing a product expressed by the three-dimensional information;

a generating unit that, if the inspecting unit detects an inexpedient part, generates inexpedient information for three-dimensionally expressing the inexpedient part; and

an outputting unit that outputs the inexpedient information in association with an inspection item.

(A2) The information processing apparatus described in (A1), in which

the outputting unit outputs three-dimensional information for displaying including the inexpedient part by using the three-dimensional shape information received by the receiving unit and the inexpedient information.

(A3) The information processing apparatus described in (A1) or (A2), in which

the inspecting unit further performs a mold requirement inspection for at least one of a thick wall, a thin wall, a mold thin wall, a product edge, a mold edge, and a snap fit, as the mold requirement inspection performed by the inspecting unit.

(A4) The information processing apparatus described in any of (A1) to (A3), further including:

a specifying unit that specifies a mold opening direction from the three-dimensional information, in which

the inspecting unit performs an inspection based on the specified mold opening direction.

(A5) The information processing apparatus described in any of (A1) to (A4), in which

the receiving unit receives an inspection item to be performed by the inspecting unit, and

if the inspection item is received by the receiving unit, the inspecting unit performs a mold requirement inspection corresponding to the inspection item.

(A6) The information processing apparatus described in any of (A1) to (A5), in which the receiving unit receives a value corresponding to an inspection item to be performed by the inspecting unit, and

if the value is received by the receiving unit, the inspecting unit performs a mold requirement inspection for the corresponding inspection item in accordance with the value.

(A7) The information processing apparatus described in any of (A1) to (A6), in which

the receiving unit receives a mold opening direction expressed by the three-dimensional information, and

if the mold opening direction expressed by the three-dimensional information is received by the receiving unit, the inspecting unit performs a mold requirement inspection in accordance with the received opening direction.

(A8) An information processing program causing a computer to function as units, the units including:

a receiving unit that receives three-dimensional information including at least a three-dimensional shape;

an inspecting unit that performs a mold requirement inspection including at least an undercut on the three-dimensional information, as a mold requirement inspection for manufacturing a product expressed by the three-dimensional information;

a generating unit that, if the inspecting unit detects an inexpedient part, generates inexpedient information for three-dimensionally expressing the inexpedient part; and

an outputting unit that outputs the inexpedient information in association with an inspection item.

(1B) An information processing apparatus including:

an inspecting unit that performs a mold requirement inspection on three-dimensional information for manufacturing a product expressed by the three-dimensional information; and

an outputting unit that outputs information for plotting a line indicative of an inexpedient part in a space configured of a surface of the product expressed by the three-dimensional information, as information indicative of an inspection result by the inspecting unit.

(2B) The information processing apparatus described in (1B), in which

the outputting unit outputs information for plotting a surface being an inexpedient part of the product expressed by the three-dimensional information, in a manner distinguishable from a surface not being an inexpedient part.

(3B) The information processing apparatus described in (1B) or (2B), in which

the inspecting unit performs a mold requirement inspection for at least one of an undercut, a thick wall, a thin wall, a mold thin wall, a product edge, a mold edge, and a snap fit, as the mold requirement inspection performed by the inspecting unit, and

the outputting unit outputs information for plotting a line indicative of an inexpedient part in a space configured of a surface of the product expressed by the three-dimensional information if the inspecting unit performs the mold requirement inspection for any of the undercut, the mold thin wall, and the snap fit.

(4B) The information processing apparatus described in (2B), in which

the outputting unit outputs information for plotting a surface being an inexpedient part of the product expressed by the three-dimensional information in a manner distinguishable from a surface not being an inexpedient part, and information for plotting a line indicative of an inexpedient part in a space configured of a surface of the product expressed by the three-dimensional information if the inspecting unit performs a mold requirement inspection for any of an undercut and a mold thin wall.

(5B) An information processing program causing a computer to function as units, the units including:

an inspecting unit that performs a mold requirement inspection on three-dimensional information for manufacturing a product expressed by the three-dimensional information; and

an outputting unit that outputs information for plotting a line indicative of an inexpedient part in a space configured of a surface of the product expressed by the three-dimensional information, as information indicative of an inspection result by the inspecting unit.

The described program may be stored in a storage medium and provided. Alternatively, the program may be provided by a communication measure. In this case, for example, the above-described program may be interpreted as an aspect of the invention of “a computer-readable medium storing a program.”

“The computer-readable medium storing the program” represents a computer-readable medium storing a program, the medium which is used for, for example, installation and execution of the program, and distribution of the program.

For example, the storage medium may include a digital versatile disk (DVD), particularly, “DVD-R, DVD-RW, DVD-RAM, and the like” complying with the standard formulated by the DVD forum, “DVD+R, DVD+RW, and the like” complying with the standard formulated as DVD+RW; a compact disc (CD), particularly, a compact disc read only memory (CD-ROM), a compact disc recordable (CD-R), a compact disc rewritable (CD-RW), and the like; a Blu-ray (registered trademark) disc; a magneto-optical disk (MO); a flexible disk (FD); a magnetic tape; a hard disk; a read only memory (ROM); an electrically erasable programmable ROM (EEPROM, registered trademark); a flash memory; a random access memory (RAM); a secure digital (SD) memory card; and the like.

The above-described program or a part of the program may be recorded in the storage medium, and may be stored and distributed. Also, the above-described program or a part of the program may be transmitted by using a wired network, a wireless communication network, a transmission medium with a combination of the wired network and the wireless communication network, used for a local area network (LAN), a metropolitan area network (MAN), a wide area network (WAN), the Internet, an intranet, an extranet, or the like; or may be carried on a carrier wave.

Further, the program may be a part of other program, or may be recorded in a storage medium together with a different program. Alternatively, the program may be divided and recorded in plural recording media. Also, the program may be recorded in any form, for example, a compressed form or an encrypted form, as long as the program may be restored.

The foregoing description of the exemplary embodiment of the present invention has been provided for the purposes of illustration and description. It is not intended to be exhaustive or to limit the invention to the precise forms disclosed. Obviously, many modifications and variations will be apparent to practitioners skilled in the art. The embodiment was chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An information processing apparatus comprising:
an inspecting unit that performs a mold requirement inspection on three-dimensional information for manufacturing a product expressed by the three-dimensional information; and
an outputting unit that outputs information for plotting a line indicative of an inexpedient part on a surface of the product expressed by the three-dimensional information, as information indicative of an inspection result by the inspecting unit.

2. The information processing apparatus according to claim 1,
wherein the outputting unit outputs information for plotting a surface being an inexpedient part of the product expressed by the three-dimensional information, in a manner distinguishable from a surface not being an inexpedient part.

3. The information processing apparatus according to claim 1,
wherein the inspecting unit performs a mold requirement inspection for at least one of an undercut, a thick wall, a thin wall, a mold thin wall, a product edge, a mold edge, and a snap fit, as the mold requirement inspection performed by the inspecting unit, and
wherein the outputting unit outputs information for plotting a line indicative of an inexpedient part on a surface of the product expressed by the three-dimensional information if the inspecting unit performs the mold requirement inspection for any of the thick wall, the thin wall, the product edge, and the mold edge.

4. The information processing apparatus according to claim 2,
wherein the inspecting unit performs a mold requirement inspection for at least one of an undercut, a thick wall, a thin wall, a mold thin wall, a product edge, a mold edge, and a snap fit, as the mold requirement inspection performed by the inspecting unit, and
wherein the outputting unit outputs information for plotting a surface being an inexpedient part of the product expressed by the three-dimensional information in a manner distinguishable from a surface not being an inexpedient part, and information for plotting a line indicative of an inexpedient part on a surface of the product expressed by the three-dimensional information if the inspecting unit performs the mold requirement inspection for the thin wall.

5. A non-transitory computer readable medium storing a program causing a computer to execute a process for information processing, the process comprising:
performing a mold requirement inspection on three-dimensional information for manufacturing a product expressed by the three-dimensional information; and
outputting information for plotting a line indicative of an inexpedient part on a surface of the product expressed by the three-dimensional information, as information indicative of an inspection result of the mold requirement inspection.

6. An information processing method comprising:
performing a mold requirement inspection on three-dimensional information for manufacturing a product expressed by the three-dimensional information; and
outputting information for plotting a line indicative of an inexpedient part on a surface of the product expressed by the three-dimensional information, as information indicative of an inspection result of the mold requirement inspection.
